(19) United States
(12) Patent Application Publication
(10) Pub. No.: US 2018/0011263 A1
(43) Pub. Date: Jan. 11, 2018

(54) OPTICAL TRANSMISSION MODULE AND ENDOSCOPE

(71) Applicant: OLYMPUS CORPORATION, Tokyo (JP)

(72) Inventor: Keiichi KOBAYASHI, Kamiina-gun (JP)

(73) Assignee: OLYMPUS CORPORATION, Tokyo (JP)

(21) Appl. No.: 15/711,275

(22) Filed: Sep. 21, 2017

Related U.S. Application Data
(63) Continuation of application No. PCT/JP2015/059210, filed on Mar. 25, 2015.

(51) Int. Cl. G02B 6/42; A61B 1/00; A61B 1/04; G02B 23/24; G02B 6/26

(52) U.S. Cl. CPC G02B 6/4202; A61B 1/04; G02B 23/2423; A61B 1/00013; G02B 6/4256; G02B 6/26

(57) ABSTRACT

An optical transmission module is configured such that: a first optical device is provided on an upper surface of an optical waveguide substrate; a second optical device is provided on a lower surface of the optical waveguide substrate; a V-groove is formed on an end face of the optical waveguide substrate, the V-groove including a first reflective face and a second reflective face as wall faces; the first optical device is optically coupled with an optical waveguide via the first reflective face; and the second optical device is optically coupled with the optical waveguide via the second reflective face.

### CROSS REFERENCE TO RELATED APPLICATION

This application is a continuation application of PCT/JP2015/059210 filed on Mar. 25, 2015, the entire contents of which are incorporated herein by this reference.

### BACKGROUND OF THE INVENTION

### 1. Field of the Invention

The present invention relates to an optical transmission module including a wiring board mounted with an optical device thereon, a waveguide substrate, and an optical fiber, and an endoscope having the optical transmission module.

### 2. Description of the Related Art

An electronic endoscope has an image pickup device such as a CCD at a distal end portion of an elongated insertion portion. In recent years, the use of high-resolution image pickup device to an endoscope has been discussed. Since when a high-resolution image pickup device is used, image signals to be transmitted from the image pickup device to a signal processing apparatus (processor) increase, there is preferably employed optical signal transmission via a thin optical fiber by an optical signal instead of electric signal transmission via a metal wiring by an electric signal. An optical transmission module for converting an electric signal to an optical signal is employed for the optical signal transmission.

Herein, optical signals with different wavelengths are superimposed, and thus an image signal with a larger capacity can be transmitted. Further, bidirectional transmission enables not only an image signal from the image pickup device to the signal processing apparatus but also clock signals and the like from the signal processing apparatus to the image pickup device to be transmitted via one optical fiber. An optical transmission module having a wave coupling/branching function is used for superimposing optical signals.

JP 2004-170668 A discloses an optical transmission module having a wave coupling function in which an optical waveguide is branched into a first waveguide and a second waveguide at a Y branch part and an optical signal is reflected on a 45-degree tilted face formed relative to an end face of a substrate so that two optical devices are arranged to be orthogonal to the waveguides.

JP 2013-142717 A discloses a polymer-type optical waveguide substrate in which a core as optical waveguide is tapered.

### SUMMARY OF THE INVENTION

An optical transmission module according to an embodiment of the present invention includes a first optical device for transmitting or receiving a first optical signal, a second optical device for transmitting or receiving a second optical signal, a polymer-type optical waveguide substrate provided with an optical waveguide for guiding a third optical signal in which the first optical signal and the second optical signal are coupled, and an optical fiber optically coupled with the optical waveguide, wherein the first optical device is provided on an upper surface of the optical waveguide substrate, and the second optical device is provided on a lower surface of the optical waveguide substrate.

An endoscope according to another embodiment includes an optical transmission module at a distal end portion of an insertion portion, the optical transmission module including a first optical device for transmitting or receiving a first optical signal, a second optical device for transmitting or receiving a second optical signal, a polymer-type optical waveguide substrate provided with an optical waveguide for guiding a third optical signal in which the first optical signal and the second optical signal are coupled, and an optical fiber optically coupled with the optical waveguide, wherein the first optical device is provided on an upper surface of the optical waveguide substrate, and the second optical device is provided on a lower surface of the optical waveguide substrate.

### BRIEF DESCRIPTION OF THE DRAWINGS

FIG. 1 is a perspective view of an optical transmission module according to an embodiment.

FIG. 2 is a cross-section view of the optical transmission module according to the embodiment.

FIG. 3A is a cross-section view for explaining a method for manufacturing the optical transmission module according to the embodiment.

FIG. 3B is a cross-section view for explaining the method for manufacturing the optical transmission module according to the embodiment.

FIG. 3C is a cross-section view for explaining the method for manufacturing the optical transmission module according to the embodiment.

FIG. 3D is a cross-section view for explaining the method for manufacturing the optical transmission module according to the embodiment.

FIG. 3E is a cross-section view for explaining the method for manufacturing the optical transmission module according to the embodiment.

FIG. 3F is a cross-section view for explaining the method for manufacturing the optical transmission module according to the embodiment.

FIG. 4A is a cross-section view for explaining the method for manufacturing the optical transmission module according to the embodiment.

FIG. 4B is a cross-section view for explaining the method for manufacturing the optical transmission module according to the embodiment.

FIG. 4C is a cross-section view for explaining the method for manufacturing the optical transmission module according to the embodiment.

FIG. 4D is a cross-section view for explaining the method for manufacturing the optical transmission module according to the embodiment.

FIG. 5 is a cross-section view of an optical transmission module according to a first modification.

FIG. 6 is a cross-section view of an optical transmission module according to a second modification.

FIG. 7 is a cross-section view of an optical transmission module according to a third modification.

FIG. 8 is a cross-section view of an optical transmission module according to a fourth modification.

FIG. 9 is an outer appearance view of an endoscope according to a second embodiment.

### DETAILED DESCRIPTION OF THE PREFERRED EMBODIMENT(S)

### First Embodiment

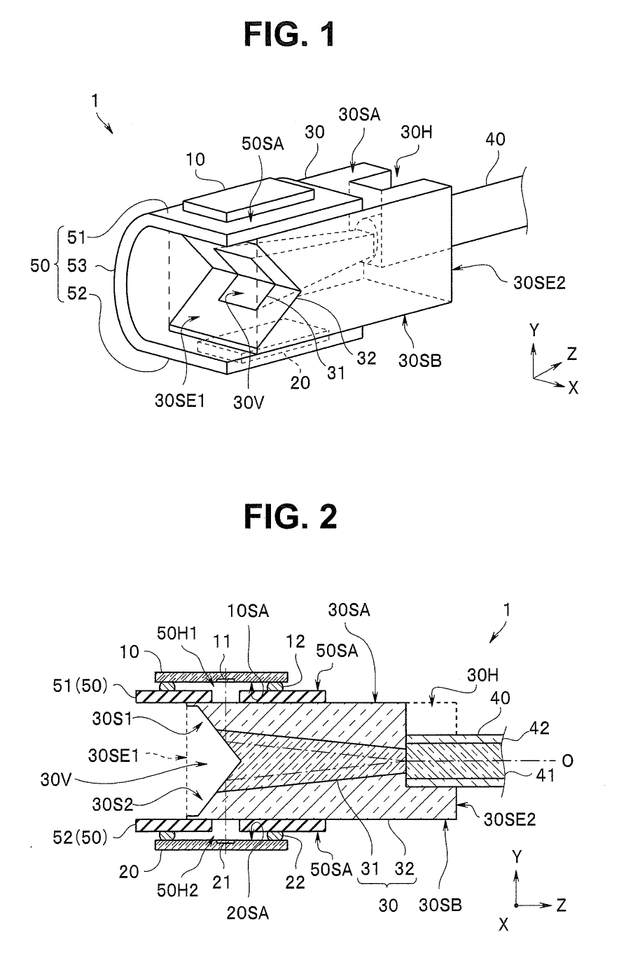

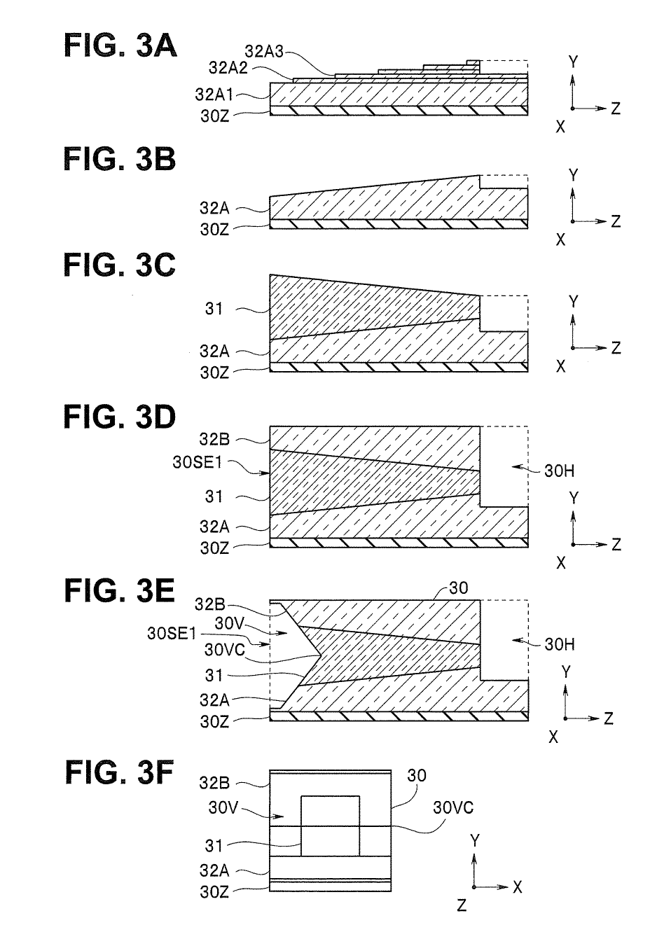

FIG. 1 and FIG. 2 illustrate an optical transmission module 1 according to an embodiment of the present invention. In the following description, it is noted that the figures according to each embodiment are schematic and a relationship between thickness and width in each part, and a ratio of thicknesses in respective parts, and the like are different from actual ones, and a relationship or ratio of mutual dimensions may be different between the figures. Further, some components may not be illustrated. For example, a support substrate 30Z (see FIG. 3A and others) may not be illustrated.

The optical transmission module 1 having a wave coupling function includes a light emitting device 10 as first optical device, a light emitting device 20 as second optical device, an optical waveguide substrate 30, an optical fiber 40, and a wiring board 50.

The light emitting device 10 transmits a first optical signal with a first wavelength λ1. The light emitting device 20 transmits a second optical signal with a second wavelength λ2 different from the first wavelength λ1.

The light emitting devices 10 and 20 are a vertical cavity surface emitting laser (VCSEL), and output a light of an optical signal in the vertical direction (Z-axis direction) relative to a light emitting face (XY plane) depending on an input electric signal. For example, the micro-light emitting devices 10, 20 in a plan view dimension of 250 μm×300 μm have the light emitting parts 11, 21 with a diameter of 20 μm at the light emitting face, respectively. For example, the first wavelength λ1 is 670 nm and the second wavelength λ2 is 850 nm.

The optical waveguide substrate 30 is a polymer-type optical waveguide substrate having an upper surface 30SA, a lower surface 30SB, a first end face 30SE1, and a second end face 30SE2 opposing the first end face 305E1. The optical waveguide substrate 30 is such that a core 31 as optical waveguide for guiding an optical signal is provided from the first end face toward the second end face and a clad 32 with a lower refractive index than the core 31 surrounds the core 31. The optical waveguide (core 31) is tapered such that the cross-section area is smaller from the first end face 30SE1 toward the second end face 30SE2.

For example, the core 31 and the clad 32 are made of polyimide fluoride resin with a refractive index of 1.60 to 1.75 which is excellent in heat resistance, transparency, and isotropy. A difference between the refractive index of the core 31 and the refractive index of the clad 32 is preferably between 0.05 and 0.20 for efficient optical transmission.

The optical waveguide substrate 30 may be a quartz optical waveguide, but is preferably a plate-shaped polymer waveguide which is more excellent in machinability than inorganic material and can be easily manufactured at low cost.

The multimode-type optical fiber 40 is configured of the core 31 with an outer diameter of 125 μm and a diameter of 50 μm for transmitting a light, and the clad 32 covering the outer periphery of the core 31. The optical fiber 40 may be covered with a resin-made outer coat. The optical fiber 40 is inserted into a groove 30H as attachment part formed on the second end face 30SE2 of the optical waveguide substrate 30.

In the optical transmission module 1, the light emitting device 10 and the light emitting device 20 are surface-mounted on the main surfaces of the flexible wiring board 50. However, the light emitting device 10 is provided on the upper surface 30SA of the optical waveguide substrate 30, and the light emitting device 20 is provided on the lower surface 30SB. Assuming the increasing direction on the X axis as upward direction, the light emitting device 10 and the light emitting device 20 are provided on the side faces of the optical waveguide substrate 30.

The flexible wiring board 50 is configured of a first plate part 51 which is mounted with the light emitting device 10 and bonded on the upper surface 30SA, a second plate part 52 which is mounted with the light emitting device 20 and bonded on the lower surface 30SB, and a bent part 53 between the first plate part 51 and the second plate part 52. Then, a sum of an angle formed between the first plate part 51 and the bent part 53 and an angle formed between the second plate part 52 and the bent part 53 is 180 degrees.

The flexible wiring board 50 is mainly made of polyimide or the like, and has the connection terminals on which the light emitting device 10 and the light emitting device 20 are surface-mounted on a main surface 50SA. Further, the wiring board 50 is formed with a throughhole 50H1 as optical path for the first optical signal transmitted by the light emitting device 10, and a throughhole 50H2 as optical path for the second optical signal transmitted by the light emitting device 20.

Then, a V-groove 30V parallel with the X axis is formed on the first end face 30SE1 of the optical waveguide substrate 30. The wall faces of the V-groove 30V configure a first reflective face 30S1 and a second reflective face 30S2. That is, the first reflective face 30S1 and the second reflective face 30S2 are integrally formed.

The inside of the V-groove 30V is a cavity in FIG. 2 and others, but may be filled with resin. Further, a reflective film such as gold film may be formed on the first reflective face 30S1 and the second reflective face 30S2.

The light emitting device 10 is optically coupled with the optical waveguide (core 31) via the first reflective face 30S1, and the light emitting device 20 is optically coupled with the optical waveguide (core 31) via the second reflective face 30S2.

That is, the first optical signal transmitted by the light emitting device 10 and the second optical signal transmitted by the light emitting device 20 are coupled while passing through the core 31 of the optical waveguide substrate 30, thereby being guided as third optical signal to the optical fiber 40.

The first optical signal and the second optical signal are coupled by the V-groove 30V, and thus the optical transmission module 1 is short in its entire length and transverse width. Further, the light emitting device 10 and the light emitting device 20 are provided on and below the optical waveguide substrate 30, respectively, and thus the transverse width is much shorter. Further, the distances between the light emitting devices 10, 20 and the core 31 are short, and thus transmission efficiency is excellent.

### <Method for Manufacturing Optical Transmission Module 1>

A method for manufacturing the optical transmission module 1 will be described below.

As illustrated in FIG. 3A, the lower clad sheets 32AS1, 32A2, and 32A3 are sequentially laminated on the support substrate 30Z. The lower clad sheets 32AS1, 32A2, and 32A3 will be denoted as lower clad sheet 32AS, respectively, below. The sizes (areas) of the laminated lower clad sheets 32AS are designed to be gradually smaller depending on the shape of the lower surface of the core 31.

The lower clad sheet 32AS is a film made of second resin with a lower refractive index than the first resin making the core 31.

As illustrated in FIG. 3B, the steps between the lower clad sheets 32AS laminated by a heating processing are eliminated to be a lower clad 32A. The leveling processing may be performed each time a lower clad sheet 32AS is laminated. Further, the lower clad sheets 32AS are thinned, which eliminates the need of the leveling processing.

As illustrated in FIG. 3C, a plurality of core sheets are laminated and the leveling processing is performed so that the core 31 is formed.

As illustrated in FIG. 3D, a plurality of upper clad sheets are laminated and the leveling processing is performed so that an upper clad 32B is formed. The upper clad 32B is provided to surround the core 31.

As illustrated in FIG. 3E, the groove 30V is formed from the end face 30SE1 in a cutting processing by a dicing blade. The groove 30V is formed such that the base 30VC is at the center of the core 31 in the vertical direction (Y-axis direction). FIG. 3F is a side view when the optical waveguide substrate 30 is observed from the end face 30SE1.

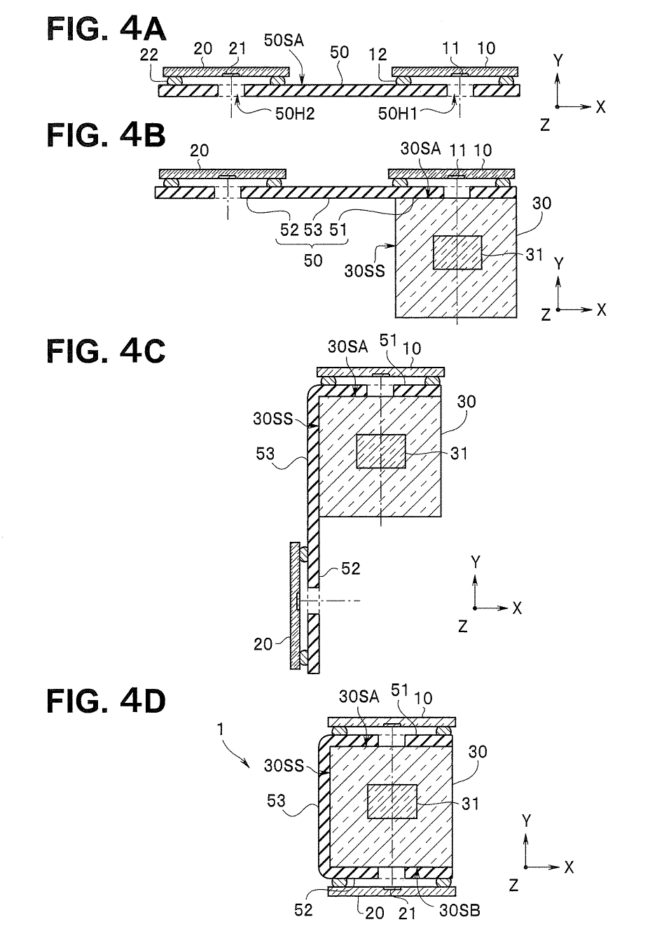

As illustrated in FIG. 4A, the light emitting devices 10 and 20 are additionally surface-mounted on the main surface 50SA of the wiring board 50. That is, the light emitting device 10 is flip-chip mounted on the wiring board 50 while the light emitting part 11 is arranged to oppose the throughhole 51H1. The light emitting device 20 is flip-chip mounted while the light emitting part 21 is arranged to oppose the throughhole 51H2.

For example, the Au bumps as connection terminals 12 of the light emitting device 10 are ultrasonically bonded with electrode pads (not illustrated) of the wiring board 50. A sealing agent such as underfill material or side-fill material may be injected into the bonded parts. After solder paste or the like is printed on the wiring board 50 and the light emitting device 20 is arranged at a predetermined position, the solder may be melted and mounted by reflow or the like. Similarly, the Au bumps as connection terminals 22 of the light emitting device 20 are ultrasonically bonded with the wiring board 50.

As illustrated in FIG. 4B, the first plate part 51 of the wiring board 50 is bonded on the upper surface 30SA of the optical waveguide substrate 30 via an adhesive (not illustrated) while the light emitting part 11 is arranged to oppose the core 31. The kind of the bonding layer is not particularly limited, but may preferably employ prepreg, buildup material, various adhesives used for manufacturing an electric wiring board, double-sided tape, ultraviolet cure adhesive, or thermosetting adhesive.

The optical waveguide substrate 30 is schematically illustrated in FIG. 4B to FIG. 4D.

As illustrated in FIG. 4C, the wiring board 50 is bent and the bent part 53 is bonded to the side face 30SS of the optical waveguide substrate 30. As illustrated in the perspective view of FIG. 1, the bent part 53 may be configured in an arc-shaped curve without tightly bonding to the side face 30SS of the optical waveguide substrate 30. With such a configuration, the light emitting part 21 and the core 31 can be easily arranged to oppose each other after the light emitting part 11 and the core 31 are arranged to oppose each other.

As illustrated in FIG. 4D, the second plate part 52 is bonded to the lower surface 30SB of the optical waveguide substrate 30 while the wiring board 50 is further bent and the light emitting part 21 is arranged to oppose the core 31. That is, the first plate part 51 and the second plate part 52 are arranged in parallel. The description is made assuming that the wiring board 50 is formed of the first plate part 51, the second plate part 52, and the bent part 53 for convenience, but the boundaries therebetween are not clearly defined.

Thereafter, though not illustrated, the optical fiber 40 is inserted into the groove 30H to be fixed by an adhesive.

For the order in which the wiring board 50 is bent, the first plate part 51 and the second plate part 52 may be bonded after the bent part 53 is bonded to the side face 30SS of the optical waveguide substrate 30.

The wiring board 50 mounted with the light emitting devices 10 and 20 thereon is bent and bonded to the optical waveguide substrate 30, and thus the optical transmission module 1 is easy to manufacture.

### MODIFICATIONS

The optical transmission modules 1A to 1D according to a first to fourth modifications will be described below. The optical transmission modules 1A to 1D are similar to the optical transmission module 1, and have the effects of the optical transmission module 1. Thus, the components having the same functions are denoted with the same reference numerals, and only the different components will be described.

### First Modification

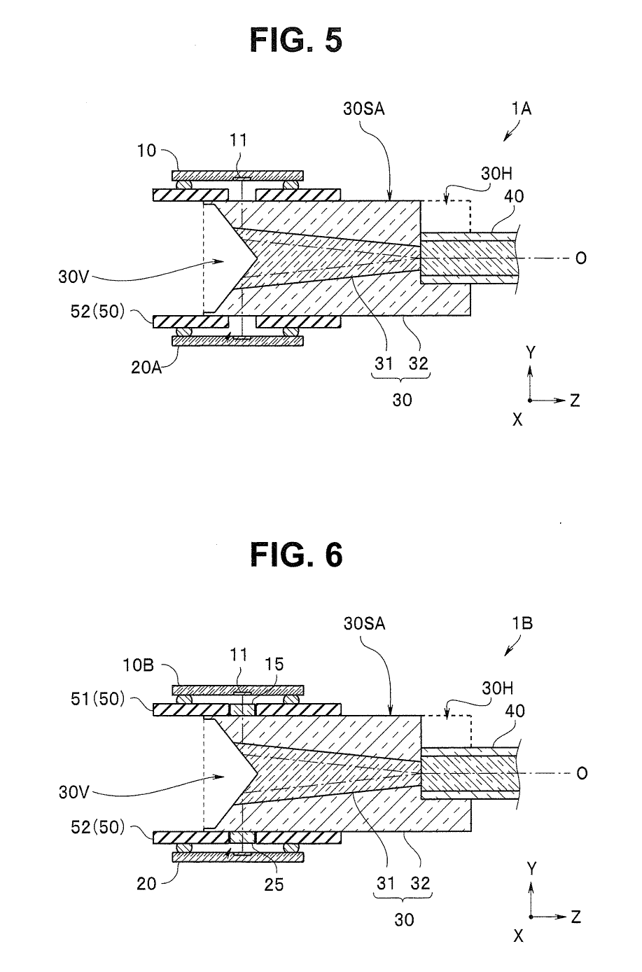

In the optical transmission module 1A according to the first modification illustrated in FIG. 5, the first optical device is the light emitting device 10 and the second optical device is a light receiving device 20A.

The light receiving device 20A is formed of a photodiode (PD), and converts and an optical signal incident into a light receiving face in the vertical direction (Z-axis direction) to an electric signal and outputs the electric signal. For example, the micro-light receiving device 20A in a plan view dimension of 250 μm×300 μm has a light receiving part 21A with a diameter of 50 μm on the light receiving face.

In the optical transmission module 1A, the first optical signal generated by the light emitting device 10 is guided via the optical fiber 40. On the other hand, the second optical signal guided via the optical fiber 40 is received by the light receiving device 20A. That is, the optical fiber 40 guides a third optical signal in which the first optical signal and the second optical signal are coupled.

### Second Modification

In the optical transmission module 1B according to the second modification illustrated in FIG. 6, the first optical device is a light receiving device 10B, and the second optical device is the light receiving device 20A. A light receiving device 10A and the light receiving device 10B may have the same or different light receiving wavelengths.

With the same light receiving wavelength, as illustrated in FIG. 6, the filters 15 and 25 are provided. For example, the filter 15 is a bandpass filter made of a dielectric multilayer film for selectively transmitting a light with the first wavelength λ1. On the other hand, the filter 25 is a bandpass filter for selectively transmitting a light with the second wavelength λ2.

For the third optical signal in which the first optical signal and the second optical signal guided by the optical fiber 40 are coupled, the first optical signal is converted to an electric signal by the light receiving device 10A, and the second optical signal is converted to an electric signal by the light receiving device 10B. That is, the optical transmission module 1B has a wave branching function.

### Third Modification

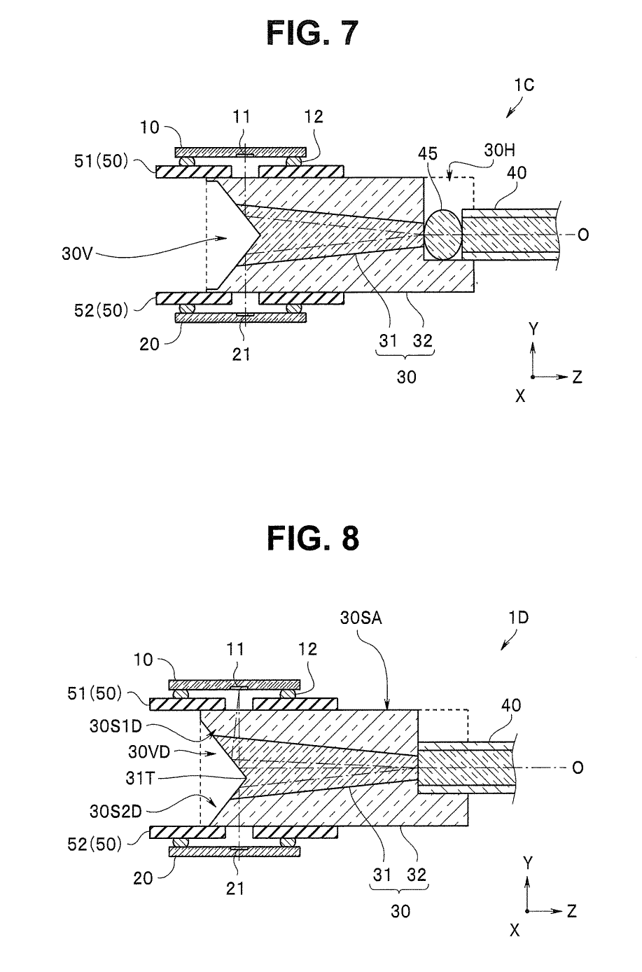

In the optical transmission module 1C according to the third modification illustrated in FIG. 7, a lens 45 as optical member for converging lights is provided between the optical fiber 40 and the optical waveguide (core 31).

For example, the transparent ball-shaped lens 45 is provided in the groove 30H while being bonded at a distal end portion of the optical fiber 40, for example.

The optical transmission module 1C having the lens 45 is strong in optical coupling between the optical fiber 40 and the core 31, which causes a small transmission loss.

### Fourth Modification

In the optical transmission module 1D according to the fourth modification illustrated in FIG. 8, the base 31T of a V-groove 30VD is offset from the center of the optical waveguide (core 31) in the vertical direction (Y-axis direction). Thus, the cross-section area of the first optical path of the light emitting device 10 is different from the cross-section area of the second optical path of the light emitting device 20.

For example, even when the intensity of the first optical signal transmitted by the light emitting device 10 is lower than the intensity of the second optical signal transmitted by the light emitting device 20, the cross-section area of the first optical path is larger, and thus the first optical signal can be efficiently transmitted.

Further, when a light receiving device and a light emitting device are provided, the cross-section area of the optical path of the light receiving device is set to be larger than the cross-section area of the optical path of the light emitting device, thereby compensating for a difference between the light receiving efficiency and the light emitting efficiency of the optical devices. That is, even when an electric signal generated by the light receiving device is small, more lights can be received.

With the optical transmission module 1D, only the base 31T of the V-groove 30VD is changed thereby to adjust the efficiency of the first optical device and the efficiency of the second optical device. Thus, it is possible to easily manufacture various optical transmission modules depending on intended use.

### Second Embodiment

An endoscope 9 according to a second embodiment will be described below.

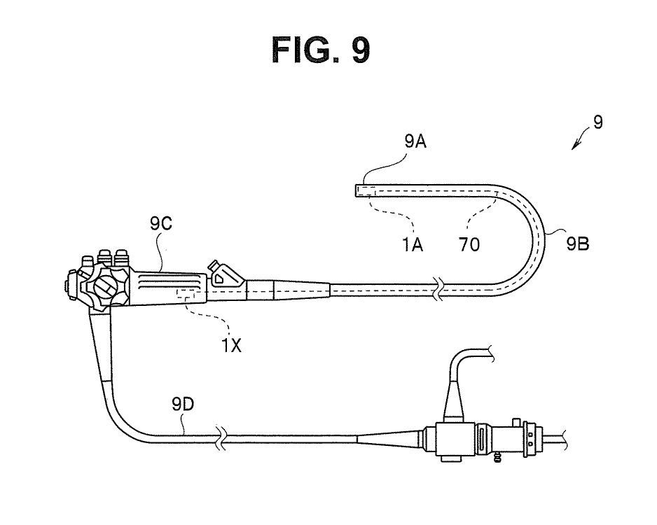

As illustrated in FIG. 9, the endoscope 9 includes an insertion portion 9B having the optical transmission module 1A provided at a distal end portion 9A, an operation portion 9C provided at the base of the insertion portion 9B, and a universal cord 9D extending from the operation portion 9C. An optical signal, which is originated from the optical transmission module 1A provided at the distal end portion 9A and guided by an optical fiber 70 inserted through the insertion portion 9B, is converted to an electric signal by an optical transmission module 1X provided at the operation portion 9C. Further, an optical signal, which is originated from the optical transmission module 1X provided at the operation portion 9C and guided by the optical fiber 70 inserted through the insertion portion 9B, is converted to an electric signal by the optical transmission module 1A provided at the distal end portion 9A.

The endoscope 9 has the small optical transmission module 1A, and thus the distal end portion 9A has a small diameter.

The present invention is not limited to the above embodiments and modifications, and can be variously changed, combined, and applied within the scope without departing from the spirit of the invention.

What is claimed is:

1. An optical transmission module comprising:
a first optical device for transmitting or receiving a first optical signal;
a second optical device for transmitting or receiving a second optical signal;
an optical waveguide substrate provided with an optical waveguide for guiding a third optical signal in which the first optical signal and the second optical signal are coupled; and
an optical fiber optically coupled with the optical waveguide,
wherein the first optical device is provided on an upper surface of the optical waveguide substrate,
the second optical device is provided on a lower surface of the optical waveguide substrate,
a first reflective face and a second reflective face are formed relative to an end face of the optical waveguide substrate,
the first optical device is optically coupled with the optical waveguide via the first reflective face,
the second optical device is optically coupled with the optical waveguide via the second reflective face, and
the first reflective face and the second reflective face are the wall faces of a V-groove formed on the end face of the optical waveguide substrate.

2. The optical transmission module according to claim 1, comprising:
a flexible wiring board whose main surfaces are mounted with the first optical device and the second optical device,
wherein the wiring board is formed of a first plate part on which the first optical device is mounted and which is bonded to the upper surface, a second plate part on which the second optical device is mounted and which is bonded to the lower surface, and a bent part between the first plate part and the second plate part, and
a sum of an angle formed between the first plate part and the bent part and an angle formed between the second plate part and the bent part is 180 degrees.

3. The optical transmission module according to claim 1,
wherein the optical waveguide is tapered.

4. The optical transmission module according to claim 1,
wherein an optical member for converging lights is provided between the optical fiber and the optical waveguide.

5. The optical transmission module according to claim 1,
wherein a base of the V-groove is offset from a center of the optical waveguide.

6. The optical transmission module according to claim 1,
wherein the first optical device and the second optical device are each a light emitting device.

7. The optical transmission module according to claim 1,
wherein the first optical device is a light emitting device and the second optical device is a light receiving device.

8. The optical transmission module according to claim 1,
wherein the first optical device and the second optical device are each a light receiving device.

9. An endoscope comprising the optical transmission module according claim 1 at a distal end portion of an insertion portion.
